(19) United States
(12) Patent Application Publication
(10) Pub. No.: US 2007/0154511 A1
(43) Pub. Date: Jul. 5, 2007

(54) Retention of endothelial cells on vascular grafts

(76) Inventors: Venkatram P. Shastri, Nashville, TN (US); Keith J. Gooch, Media, PA (US); Jason W. Nichol, Somerville, MA (US)

Correspondence Address: RATNERPRESTIA, P O BOX 980, VALLEY FORGE, PA, 19482-0980, US

(21) Appl. No.: 10/561,056

(22) PCT Filed: Jun. 25, 2004
(86) PCT No.: PCT/US04/20441

§ 371 Date: Sep. 8, 2006

Related U.S. Application Data
(60) Provisional application No. 60/482,829, filed on Jun. 25, 2003.

(51) Int. Cl. A61F 2/06

(52) U.S. Cl. 424/423; 623/1.11

(57) ABSTRACT

An implantable device including a surface containing a plurality of first zones and a plurality of second zones depressed relative to the first zones so as to provide valleys below a plane defined by the first zones, wherein a first zone to second zone width ratio is non-random throughout the device; and a biologically active agent in the valley, wherein the device is adapted to be implanted within an organism such that when said surface is subjected to a flow causing a fluid-induced shear stress, the second zone has a reduced level of the fluid-induced shear stress relative to the first zone in an amount adequate to selectively retain the biologically active agent within the valley.

### CROSS-REFERENCE TO RELATED APPLICATIONS

This application claims the benefit of provisional Application No. 60/482,829, filed Jun. 25, 2003, which is incorporated herein in its entirety.

### BACKGROUND OF THE INVENTION

1. Field of Invention

This invention relates to implanted devices, and more particularly to retention and survival of endothelial cells on implanted devices.

2. Description of Related Art

Synthetic (polymeric) vascular grafts have completely revolutionized cardiovascular surgery. However, a common problem with synthetic vascular grafts is the formation of a clot or a thrombus on the inner wall of the graft. While such formation does not affect the patency and performance of grafts with large diameters (e.g. aortic grafts) due to their large cross-sectional area, grafts of a small caliber (less that 4 mm inner diameter) can get occluded (see Clark, B. C. et al., Biomat. Med. Dev. Artif. Org., (1974), 2, 379).

Endothelial cell (EC) survival and endothelialization of implanted devices is an important aspect in increasing the patency of such devices. Studies by Herring et al., established that endothelial cells (ECs) are critical in the prevention of thrombus formation. ECs possess a negatively charged surface that is thought to play a role in the repulsion of platelets (see Surgery, (1978), 84, 498). Furthermore, ECs secrete nitric oxide (NO), which has an anti-thrombogenic effect. In view of the beneficial effects of ECs, an obvious solution is the introduction of a viable endothelium onto a surface of implants. Although several methodologies have been developed to establish a viable endothelium on synthetic graft surfaces (Teflon®, Dacron®, polyurethane) including coating with ECM molecules, chemical modification of graft surface, introduction of surface porosity; the retention of the neo-endothelium in high shear stress environment of arterial circulation has proven to be a challenge. (Zilla, P. and Greisler, H. P., 1999, Tissue Engineering of Vascular Prosthetic Grafts, R. G. Landes & Co., Austin, Tex.).

Current strategies to enhance endothelial cell retention on synthetic vascular grafts made from, for example, polyurethane and expanded polytetrafluorethylene (ePTFE), involve modification of the polymer surface to enhance adhesion of the cell to the biomaterial either by adsorption of biomolecules, chemical modification of surfaces by plasma or conventional means, and covalent attachment of biomolecules and peptides [8-11]. Typically chemical modification procedures are specific to a chemical class of polymers and results in the alteration of the polymer chemistry and structure.

Thus, despite the foregoing developments, there is still a need in the art for implanted devices adapted for retention and survival of endothelial cells on their surfaces when subjected to fluid-induced shear stress.

All references cited herein are incorporated herein by reference in their entireties.

### BRIEF SUMMARY OF THE INVENTION

This invention relates to implanted devices, and more particularly to retention and survival of endothelial cells on implanted devices, e.g., polymeric vascular grafts surfaces, when subjected to high fluid-induced shear stress, e.g., arterial blood flow.

Accordingly, the invention provides an implantable device comprising: a surface containing a plurality of first zones and a plurality of second zones depressed relative to the first zones so as to provide valleys below a plane defined by the first zones, wherein a first zone to second zone width ratio is non-random throughout the device; and a biologically active agent in the valley, wherein the device is adapted to be implanted within an organism such that when said surface is subjected to a flow causing a fluid-induced shear stress, the second zone has a reduced level of the fluid-induced shear stress relative to the first zone in an amount adequate to selectively retain the biologically active agent within the valley. In certain embodiments, the depth of the valleys is at least 0.1 μm below the plane.

In certain embodiments, the device is adapted for use as a vascular graft.

Also provided is a method for producing the implantable device of the invention, said method comprising providing a substrate having a surface; providing the surface with the plurality of first zones and the plurality of second zones depressed relative to the first zones so as to provide valleys below a plane defined by the first zones, selecting the first zone to second zone width ratio throughout the device such that when the device is implanted within an organism and said surface is subjected to a flow causing a fluid-induced shear stress, the second zone has a reduced level of the fluid-induced shear stress relative to the first zone in an amount adequate to selectively retain a biologically active agent within the valley, and thereby producing the implantable device.

Further provided is method for delivering a biologically active agent to an organism, said method comprising: providing the implantable device of the invention; regulating retaining of the biologically active agent on the implanted device by adapting the surface to have areas of a reduced level of a fluid-induced shear stress via a pattern of the first zones and the second zones provided on the surface; and subjecting the device to a flow causing the fluid-induced shear stress by implanting the device within the organism, wherein the second zones have the reduced level of the fluid-induced shear stress relative to the first zones in an amount adequate to selectively release the biologically active agent on the implanted device and thereby delivering the biologically active agent to the organism.

Additionally, the improvement is provided in a method of manufacturing of an implanted device having an outer surface adapted to retain endothelial cells, the improvement comprising regulating retaining of the endothelial cells on the surface by providing the device with areas of a reduced fluid-induced shear stress via a pattern of first zones and second zones provided on the surface, wherein the second zones are depressed relative to the first zones to provide valleys below a plane defined by the surface, wherein the pattern is chosen by selecting a first zone to second zone width ratio, a geometrical shape of each of the first zone and the second zone and/or a depth of the valleys such that the second zones have the reduced level of the fluid-induced shear stress relative to the first zones in an amount adequate to retain the endothelial cells on the implanted device.

### BRIEF DESCRIPTION OF SEVERAL VIEWS OF THE DRAWINGS

The invention will be described in conjunction with the following drawings in which like reference numerals designate like elements and wherein:

FIG. 1 is a scheme illustrating the experimental set-up for the flow studies consisting of a flow chamber 1, a silicone gasket 2, a glass slide 3, a sample 4, a peristaltic pump 8, and a compliance chamber 9 (prior art). Line 6 is for the out-flow and line 7 is for the in-flow. Vacuum 5 is created in the flow chamber.

FIG. 2A is a top view of a patterned surface (a film) having plateaus and valleys.

FIG. 2B is a cross-sectional view of the patterned surface 14 of FIG. 2B having plateaus 11 and valleys comprising the bottom 12 and the sides 13.

FIG. 2C is a three-dimensional view of the patterned surface 14 of FIG. 2A.

FIG. 3 is a fluorescent image of bovine aortic endothelial cells (BAEC) stained with a nuclear stain DAPI on a patterned polyurethane surface during static conditions, wherein light portions are plateaus (P) of about the size of 90 microns and dark portions are channels (C) of about the size of 5-10 microns. This image illustrates that cells are capable of growing in such narrow channels.

FIGS. 4A and 4B are bar graphs illustrating density of BAEC on unpatterned and patterned polyurethane (PU) surface under static conditions (control) and after flow. In FIG. 4A, the bar graph illustrates BAEC density on patterned and unpatterned PU surfaces under static conditions (controls) and after exposure to a shear stress of 60 dynes/cm² for 1 hour (patterned surfaces marked with a double asterisk have p<0.005. FIG. 4B demonstrates a distribution of cell densities on unpatterned surface as well as the distribution of cell densities between plateaus and valleys in the patterned surface. Error bars represent standard deviation.

FIG. 5A is a scheme illustrating a shape of a channel (valley).

FIG. 5B is a graph demonstrating computational measurements of sheer stress values in the channel of FIG. 5A.

FIG. 6 is a fluorescent image of BAEC stained with a nuclear stain DAPI on a patterned polyurethane surface after exposure to flow. The black areas are the plateaus (P) showing the absence of cells (white dots) and rows of white dots are valleys (V). The flow was applied in the direction of the length of the valleys. The scale bar was 100 micrometers.

FIGS. 7A, 7B, 7C, and 7D are two dimensional schemes illustrating various geometries of closed valleys and their patterns on the surface of the device (valleys that are completely surrounded by at least one wall), wherein FIG. 7A is showing closed valleys in a shape of channels with both ends closed, and FIGS. 7B-7D show closed valleys in a shape of pentagons, circles and triangles.

### DETAILED DESCRIPTION OF THE INVENTION

The invention was driven by a desire to develop an implantable device capable of selective retention and release of a biologically active agent on its surface when the device is implanted in an organism and subjected to a fluid-induced shear stress. The inventors have discovered that such device can be obtained by selecting a pattern of zones with different depth, width and geometry and assessing the level of the shear stress as to select the zones in which the shear stress is less than that of the areas in which the device is implanted, e.g., an artery, to be the desired zones in which the biological agent is selectively retained and released to the organism at the place of implantation.

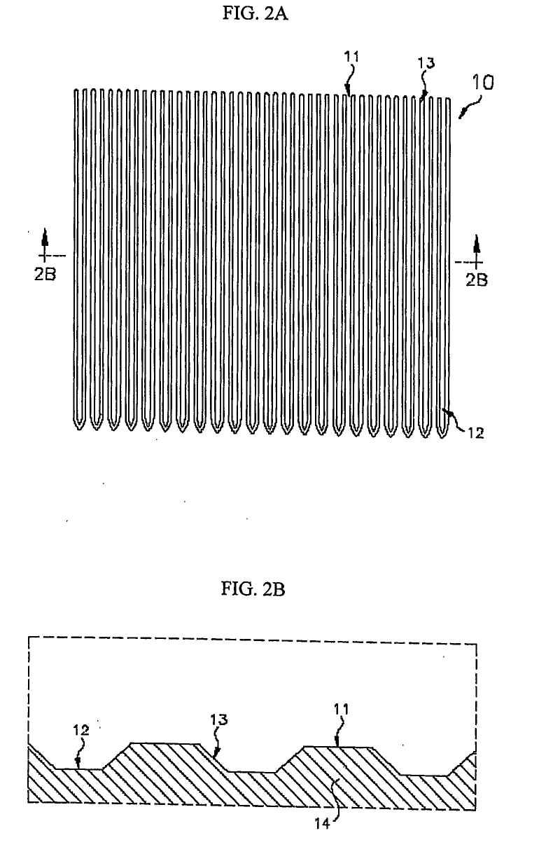

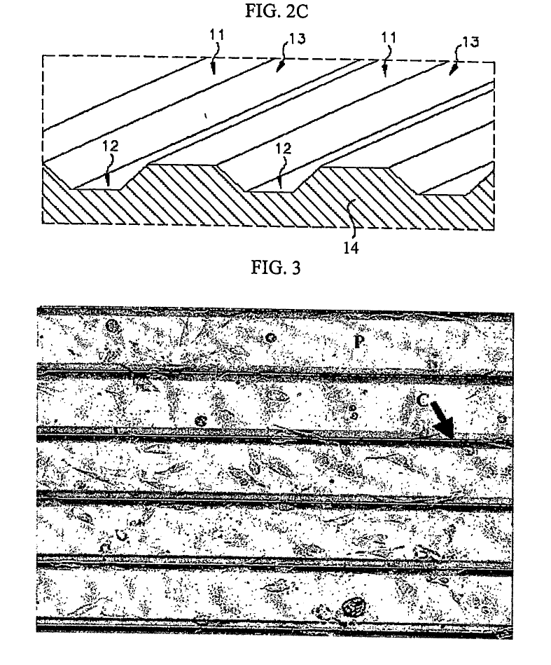

The invention addresses the problem of de-endothelialization (i.e., EC shedding) of pre-seeded synthetic vascular grafts using a non-chemical approach. Inventors discovered that by creating well-defined micropatterns on a surface, fluid flow can be altered to create discrete regions of low-no shear stress. The invention is based on the belief that due to reduced stress; EC retention in these discrete regions will be enhanced. Inventors studied the retention of bovine aortic endothelial cells (BAEC) on polyurethane (PU) surfaces that were patterned with an array of alternating micro-channels and compared them to unpatterned surfaces (see FIGS. 2A-2C). The invention demonstrates a novel approach for ensuring retention of EC's under a high shear stress of 60 dynes/cm² on synthetic polymer graft surfaces. This was achieved by creating well-defined micropatterns of valleys on a surface of a device. Fluid dynamic simulations as described in detail below suggest that under the experimental conditions, stagnation in the channel can occur leading to a low-no stress environment, which is favorable for EC retention. As shown below, with the appropriate choice of geometry of valleys and spatial distribution on the surface, high retention of ECs may be achieved.

The invention provides an implantable device comprising: a surface containing a plurality of first zones and a plurality of second zones depressed relative to the first zones so as to provide valleys below a plane defined by the first zones, wherein a first zone to second zone width ratio is non-random throughout the device; and a biologically active agent in the valley, wherein the device is adapted to be implanted within an organism such that when said surface is subjected to a flow causing a fluid-induced shear stress, the second zone has a reduced level of the fluid-induced shear stress relative to the first zone in an amount adequate to selectively retain the biologically active agent within the valley.

The term “implantable” means permanently or temporality inserted into an organism.

The term “implantable device” is a device, which is adapted for permanent or temporary insertion into or application against a tissue of an animal such as a human and is not limited to a vascular graft, a catheter, a conduit, and any flat or curved surface.

Non-limiting examples of implantable devices further include degradable and non-degradable sutures, orthopedic prostheses such as supporting rod implants, joint prostheses, pins for stabilizing fractures, bone cements and ceramics, tendon reconstruction implants, prosthetic implants, cardiovascular implants such as heart valve prostheses, pacemaker components, defibrillator components, angioplasty devices, intravascular stents, acute and in-dwelling catheters, ductus arteriosus closure devices, implants deliverable by cardiac catheters such as atrial and ventricular septal defect closure devices, urologic implants such as urinary catheters and stents, neurosurgical implants such as neurosurgical shunts, ophthalmologic implants such as lens prosthesis, thin ophthalmic sutures, and corneal implants, dental prostheses, and internal and external wound dressings such as bandages and hernia repair meshes.

Implantable devices can be made from biodegradable and non-biodegradable polymers such as, for example polyamides, polycarbonates, the polyurethane, Teflon®, Dacron® as well as metals or metal alloys, ceramics and a composition and a mixture thereof. Polymers can be applied on a surface of other polymers, metals or ceramics to create the desired geometry and patterns of plateaus and valleys as described below using methods known in the art such as, for example, microprinting, lithography and etching.

The term “biologically active agent” as used herein means an agent which can influence a biological reaction or a function of an organism. Examples of such agents are cells, pharmacological agents which have a pharmacological activity in the organism, and other agents known in the art to have such function. Preferably, the biologically active agent of the invention is an endothelial cell.

The term “first zones” means areas that are on the same level as the plane of the surface of the device; the first zones are also referred herein as “plateaus”.

The term “second zones” means zones that are depressed relatively to the plane of the surface of the device; the second zones are referred herein as “valleys” or “channels.” Second zones of the invention have a desired depth, width, and geometry selected based on desired level of sheer stress inside the valley. Also, the valleys have walls and the bottom wherein the biologically active agent is selectively retained.

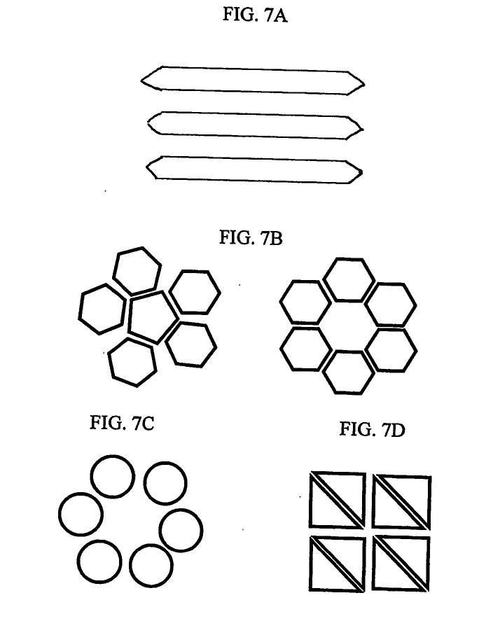

In certain embodiments, the valley is at least one of an open valley, a partially closed valley, and a closed valley. In one variant of this embodiment, the valley has one opening, i.e., a partially closed valley (as for example shown in FIG. 2, wherein one end of the valley is a closed pointed end and another end is open). In another one variant of this embodiment, the valley has two openings, i.e., an open valley, wherein the openings are preferably disposed on opposite ends of the valley, i.e., an open-ended channel. In yet another variant of this embodiment, the valley is completely surrounded by at least one wall, e.g., a half of a sphere (FIG. 7C), or two walls, e.g., a closed ended channel, to form a closed valley (FIG. 7A). Non-limiting examples of the width of the valley are at least 5 microns, at least 10 microns, at least 90 microns, and at 150 microns or less. Non-limiting examples of the depth of the valley is at least 0.1 micron.

Patterns of first zones/second zones can be the same or different throughout the surface of the device. Orientation to the channels with relation to the flow can vary in accordance with the desired applications. Preferably, the flow is directed along the length of the channel/valley, however, the valleys can also be oriented to be substantially perpendicular to the flow, and disposed at a non-parallel and non-perpendicular angle to the flow. A combination of the above directions is also useful herein. To ensure obtaining 100% coverage of the surface with the biologically active agent, the areas of low shear stress would have to be spaced very closely to each other in a dense packing on the surface of the device.

In certain embodiments, a geometrical shape of each of the first zone and the second zone and/or a depth of the valleys are non-random. Preferably, the surface of the device has patterns of the first zones and the second zones non-randomly distributed across the surface. Examples of such distribution are shown in FIGS. 7A-C and FIG. 2A.

The term “a first zone to second zone width ratio” means a ratio of the width of the first zone to the width of the second zone. As described in detail below, the valleys have a reduced level of stress relatively to the plateaus. Thus, according to the invention, it is preferred to minimize the areas of higher stress and maximize the areas of lover stress in which the biologically active agent is retained best. Thus, the first zone to second zone width ratio is an important tool for selection of the desired patterns. Accordingly, in certain embodiments, the first zone to second zone width ratio is less than 2, preferably less than 1, more preferably less than 0.5 and even more preferably less than 0.1.

In certain embodiments, the reduced level of the fluid-induced shear stress is at least 20% less than a fluid-induced shear stress of the first zone. In certain embodiments, the reduced level of the fluid-induced shear stress is about 20% to about 100% less than a fluid-induced shear stress of the first zone.

Implanted devices experience a fluid sheer stress from body liquids when inserted in a body. Depending on the area of insertion, the level of the fluid sheer stress can differ. There are methods known in the art for measuring such levels, and an average sheer stress is about 20 dyn/cm². As described in the Examples below, the samples having surfaces patterned to have a plurality of valleys and plateaus as well as control unpatterned surfaces were subjected to a much higher stress of 60 dyn/cm² for one hour to demonstrate that the invention works even at such extreme conditions.

In certain embodiments, the device of the invention is adapted to deliver the biologically active agent to the organism upon implantation. In that the biologically active agent is applied to the surface of the implantable device prior to implantation.

In certain embodiments, the device is adapted to substantially retain the biologically active agent on the device upon implantation. In certain embodiments, the device is adapted to substantially retain the biologically active agent on the device upon implantation without being covalently bound to the device. A covalent bonding or specific binding (such as, for example, ligand-substrate binding) is contemplated also as well as a combination of the above. To help the adhesion of the biologically active agent to the device, substances like fibrinogen can be used. Also, a pretreatment of surfaces using methods known in the art such as plasma etching can be used to make surfaces more susceptible to adhesion.

The method for producing the implantable device of the invention is also provided herein. The method includes providing a substrate having a surface; providing the surface with the plurality of first zones and the plurality of second zones depressed relative to the first zones so as to provide valleys below a plane defined by the first zones, selecting the first zone to second zone width ratio throughout the device such that when the device is implanted within an organism and said surface is subjected to a flow causing a fluid-induced shear stress, the second zone has a reduced level of the fluid-induced shear stress relative to the first zone in an amount adequate to selectively retain a biologically active agent within the valley, and thereby producing the implantable device. In certain embodiments of the method, the first zone to second zone width ratio is less than 2, preferably less than 1, more preferably less than 0.5 and even more preferably less than 0.1. In certain embodiments, the method further comprises selecting a geometrical shape of each of the first zone and the second zone and/or a depth of the valleys.

Using computational fluid dynamics as an exemplary tool for estimating sheer stress of a model as described in Example 3, a person skilled in the art will be able to make the device of the invention by selecting the desired geometry, width and depths of second zones and their placement within the surface using the first zone to second zone width ratio relative to the shear stress at the implantation site.

Further provided is method for delivering a biologically active agent to an organism. The method includes providing the implantable device of the invention; regulating retaining of the biologically active agent on the implanted device by adapting the surface to have areas of a reduced level of a fluid-induced shear stress via a pattern of the first zones and the second zones provided on the surface; and subjecting the device to a flow causing the fluid-induced shear stress by implanting the device within the organism, wherein the second zones have the reduced level of the fluid-induced shear stress relative to the first zones in an amount adequate to selectively release the biologically active agent on the implanted device and thereby delivering the biologically active agent to the organism. In certain embodiments of the method, regulating comprises selecting the first zone to second zone width ratio, a geometrical shape of each of the first zone and the second zone and/or a depth of the valleys. In certain embodiments of the method, the biologically active agent is a cell.

The invention will be illustrated in more detail with reference to the following Examples, but it should be understood that the present invention is not deemed to be limited thereto.

### EXAMPLES

Creating patterns of alternating plateaus (P) and valleys (V) on a surface allows for comparison between adjacent regions of differing stress and serves as a model system to demonstrate the invention.

### Example 1

### Preparation of Micro-Patterned Polyurethane (PU) Films

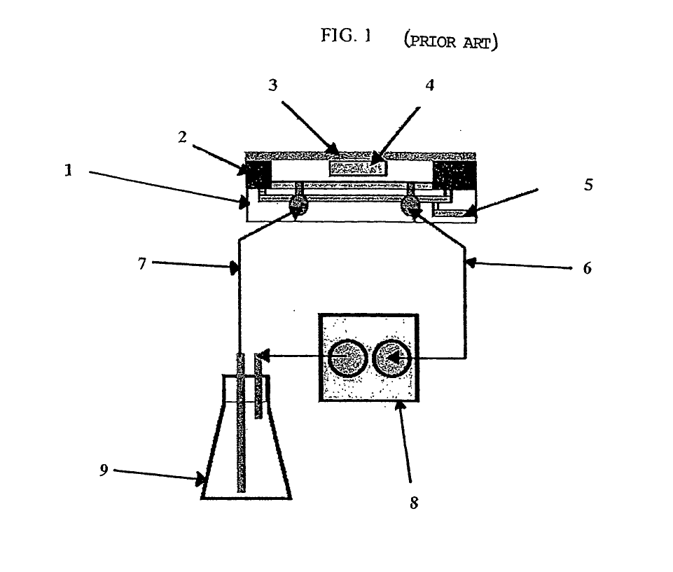

The pattern was transferred onto medical grade PU films by a solvent casting technique known in the art (see FIG. 1). In brief, a warm solution (45° C.) of PU in THF (75 mg/ml) was deposited on a silicon wafer template in a drop-wise manner until complete surface converge was achieved. The film was air-dried for 12 hours and released from the silicon substrate by soaking in isopropanol. Non-patterned PU films were made by a similar casting procedure on virgin silicon wafers. Under these conditions films of about 160 mm in thickness were obtained. The patterned films were cut into 16 mm squares; each square represents four arrays of channels (FIG. 2A-C) (each square is 4 mm×5 mm). PU films were sterilized by immersion in 70% ethanol for 30 minutes.

PU films were assembled onto to the center of sterile glass slides (#12-550B, Fisher Scientific, Hampton, N.H.) using sterile vacuum grease such that the length of the channel (major axis) was parallel to the direction of flow. The assembly was carried out in a laminar flow cell culture hood to ensure sterility. The film-slide assemblies were placed in 10 cm Petri dishes (#353003, FALCON by BD Biosciences, San Jose, Calif.) and the PU surface of patterned and non-patterned samples was coated with a 100 ml drop of fibronectin solution in 1×PBS (100 mg/ml) and air-dried for 1 hour. Once the drop dried, the PU-slide assembly was bathed in DMEM supplemented with 10% FBS and 1% penicillin/streptomycin (culture medium) and left in the incubator for 12 hours until EC seeding. Bovine aortic endothelial cells (BAEC) were isolated as described in the literature (Jaffe, A et al., 1973) and cultured in T75 flasks in culture media. P3-P5 ECs at a density of 400K cells/square were seeded 24 hours prior to the flow experiment. Unpatterned PU film and gelatin (0.1%) coated glass slides were used as static controls. ECs on all substrates exhibited cobblestone morphology typical of a confluent monolayer.

The flow studies were carried out using a closed-loop circuit comprising of a peristaltic pump 8, a compliance chamber 9, media reservoir placed in a 37° C. bath and a parallel-flow chamber 1, in series. The parallel-flow chamber 1 used in this study (see FIG. 1), produced a laminar flow (see Reich, K. M. and Frangos, J. A., 1991, Am. J. Physiol. 261 (Cell Physiol 30), C428)). PU films seeded with ECs were exposed to shear stress of ˜60 dynes/cm2 for the duration of 1 hour. Cells were fixed using 10% paraformaldehyde for 10 minutes and then mounted with mounting medium containing DAPI. Images were acquired using a Zeiss fluorescent microscope coupled to a CCD camera, with a 5× objective, using AxioPlane software. A total of 16 images were taken from the center portion of the film for each sample. A corresponding bright field image was taken to visualize channel outlines. FIG. 2A shows schematically the geometry of the patterned surface. The cells were counted manually using a transparency of bright field image as a template. The projected area in a given image was determined using the following conversion factor: 513 pixels=1 mm², which was obtained using a 5× image of a calibrated slide. The projected area for the plateau P and the valley V regions was 0.59 and 0.58 mm², respectively.

### Example 2

### Preparation of Pattern Template and Flow Studies

A negative impression of the desired pattern of alternating closed channels was created on a silicon wafer (4 cm) substrate using standard lithography techniques.

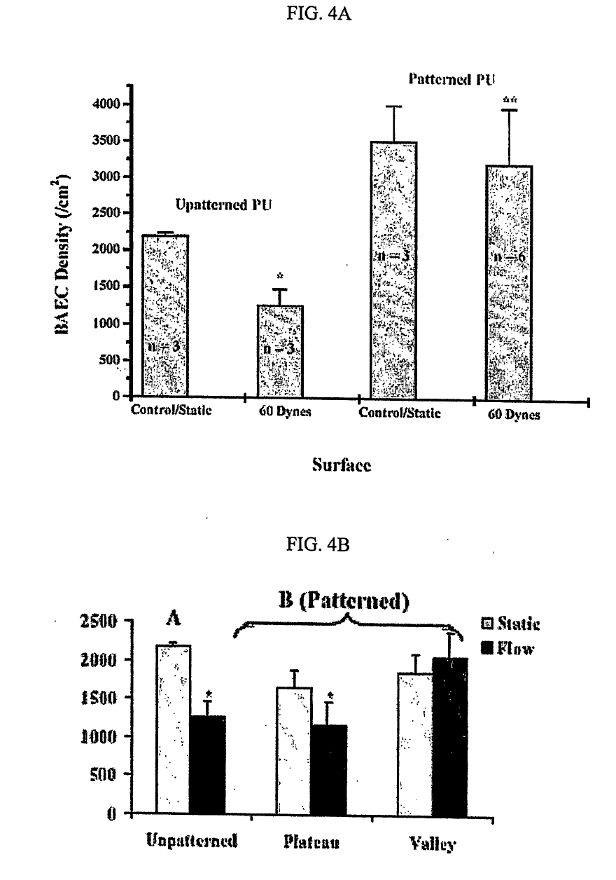

Flow studies were carried out using two different substrates: unpatterned PU and patterned PU. The results are shown in FIGS. 4A and 4B. Polymer substrates not subjected to flow “static condition” were used as positive controls. We observed that upon exposure of EC's on unpatterned surfaces to flow, density of ECs (cells/mm²) was diminished by 40±46% from 2198±37 to 1265±218 (n=3). Furthermore, de-endothelialization was observed to occur in a “patchy” manner. The difference in EC density between the static and flow conditions was statistically significant with a p<0.008.

In the case of the patterned surfaces, EC densities in the P and V regions in controls (at static conditions) were statistically identical (n=3, p=0.18) with cell densities of 1651±235 and 1864±252, respectively with an average P/V ratio of 0.89.

After exposure to flow-induced shear stress, the EC densities in the plateaus and valleys were statistically different (n=4, p<0.02) with cell densities of 1171±311 and 2056±334, respectively with an average P/V ratio of 0.56. This constitutes a 29±2% reduction in cell density in the plateaus with respect to patterned controls. In some regions on the polymer surface, a total loss of cells from the plateau region was observed with retention in corresponding valleys as shown in FIG. 6. In comparison, the EC density in the valleys after flow (2056±334) was statistically similar (n=4, p>0.2) to densities in static controls (1864±252).

In FIG. 4B, the asterisk indicates that flow decreased cell density relative to corresponding static conditions (p<0.008 for unpatterned and p<0.024 for the plateau). Error bars represent standard deviation.

### Example 3

A computation fluid dynamics (CFD) study, which includes generating desired geometries using CAD and then carrying out simulations on these geometries using CFD packages such as FemLab was conducted to study fluid sheer stress dynamics of surfaces having patterns of plateau defined by the plane of the surface (first zones) and depressions (valleys or second zones) of certain geometries, depth and height as well as their placement within the plane of the flat surface by comparison with a control model, a flat continuous surface without depressions.

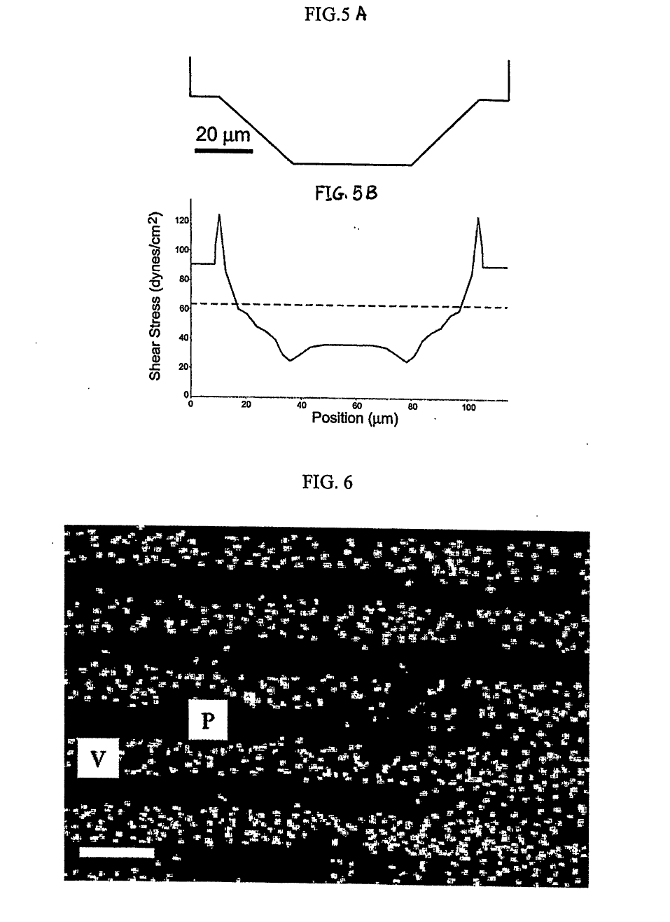

A simplified 3-D geometry was created in pro/ENGINEER 2001 version (Parametric Technology Corporation, Needham, Mass.) and imported into FEMLAB (2.3, Comsol Inc., Los Angeles, Calif.) to computationally estimate the shear stress in the valley and plateau regions of the surface made of a polymer (FIG. 5A). In the valley/plateau model, the depth in the middle of the valley or a channel was 24.5 μm high (half the total spacing, h) where the contribution of entry/exit effects were assumed to be negligible. The solution was found to not vary with length, and thus, a model length of 50 μm was used. The fluid flow was modeled as a linear variation with the maximum linear velocity determined by extrapolating the slope of the roughly linear portion of the parabolic velocity profile near the surface (from 0-10 μm) found using an average flow rate of 275 ml/min and the relationships Q=Vₐᵥₑ*h*w, and 1.5*Vₐᵥₑ=Vₘₐₓ. Other examples included geometries as shown in FIG. 2B, wherein the depth of the valley was 35 microns, the width was 94 microns, the bottom was 42 microns, the sides were at an angle of about 137.6 degrees, and plateaus were 62 microns.

To validate the method, a control model approximating a flat surface was tested, where the shear stress on the slide surface can be calculated by the relationship τ=6 Qμ/wh². The shear stress was confirmed in the control model to be within 1.6% of the analytical solution. A finite element mesh was automatically generated in FEMLAB with 611 nodes and 2206 elements for the valley/plateau model. The velocity gradient (dv/dy) was determined for both the control and valley/plateau models by solving the Navier-Stokes equations using FEMLAB's Chemical Engineering Module. The maximum fluid velocity was prescribed to the plane at the top of the model geometry, inducing a uniform linear fluid velocity field in the control model, and a velocity field that was non-linear within 100 μm of the surface of the channel model. Boundary conditions were no-slip for the solid surfaces (i.e. the polymer surface), prescribed velocity for the top surface, straight-out (open to fluid flow) for the front and rear faces, and symmetric for both side surfaces (to extend the geometry and eliminate false edge effects). From the velocity profile, the shear stress was calculated with the equation τ=−μdv/dy by measuring the distance (dy) at 0.015 m/s (dv) after thresholding, using Scionimage 4.0 (Scion Corp., Frederick, Md.). Variables that relate to fluid where as follows: Q=flow rate, V=velocity (either average (ave) or maximum (max)), μ=viscosity, dv/dy=velocity gradient and τ=shear stress. Variables relating to the flow chamber where as follows: h=height of chamber (distance between the parallel plate and the slide surface) and w=width of the chamber.

The velocity profiles for fluid flow in both the plateau/valley model (FIG. 5 A) and the control model (a flat continuous surface) were determined using FEMLAB. The velocity fields were created by prescribing a constant velocity of 1.5 m/s to the top surface of the geometry, causing a linear velocity field throughout the control model and roughly the upper half of the plateau/valley model. The solution was determined from 1-10 seconds, and found to not vary with time, suggesting the solution had reached steady state. The velocity gradient (dv/dy) was determined graphically in ScionImage by measuring the distance between the surface and the 0.015 m/s contour line at several points along the surface (FIG. 5B). Next, the shear stress was calculated for the control and the plateau/valley models.

It was observed that the average shear stress remained constant for both the control model and the plateau/valley model, the stress of the plateau regions had increased relative to the control sample and the stress of the valley regions had decreased relative to the control sample. Further, it was observed that the stress level depends on the geometry of the valley (e.g., the channel in this experiment), wherein the lowest stress level was observed between the bottom of the channel and the side wall. The average shear stress of the plateau regions (82.4 dyn/cm²) was 30.3% greater than the shear stress of the control model (63.2 dyn/cm²), while the average stress on the side walls of the valley (52.9 dyn/cm²) was reduced (16.4%). The average shear stress on the bottom surface of the valley (31.5 dyn/cm²) was 50.2% less than the shear stress of the control model. The junction between the side walls and bottom of the valley was the region with the largest velocity gradient and lowest shear stress (23.1 dyn/cm²), which was 63.5% less than the control model, while the plateau/side wall junction was the region of maximum shear stress (102.0 dyn/cm², 61.3% increase). Assuming the shear stress remains constant for the additional plateau region, the average shear stress for one entire channel/plateau was calculated by numerically determining the area under the stress/position curve, and dividing by the total length. The average shear stress of the channel/plateau was determined to be 58.3 dyn/cm², which is similar to the average of the control model (63.2 dyn/cm²).

A representative channel geometry (FIG. 5A) was modeled in FEMLAB, simulating a fluid velocity field in the direction along the length of the channel (the valley). The velocity profile was linear for the channel and plateau regions outside the boxed region. The fluid velocity profile of the boxed region was obtained for velocities ranging from 0 to 0.5 m/s (not shown). Measuring the distance from the plateau/channel surface to the contour line of 0.15 m/s, the shear stress was determined along the surface s shown in FIG. 5B.

### REFERENCES

1. Schmidt J A, von Recum A F, Macrophage response to microtextured silicone, Biomaterials, 13(15), 1059;
2. van Kooten T G, von Recum A F, Cell adhesion to textured silicone surfaces: the influence of time of adhesion and texture on focal contact and fibronectin fibril formation, Tissue Eng. 1999, 5(3), 223;
3. van Kooten T G, Whitesides J F, von Recum A, Influence of silicone (PDMS) surface texture on human skin fibroblast proliferation as determined by cell cycle analysis, J Biomed Mater Res. 1998, 43(1), 1;
4. den Braber E T, de Ruijter J E, Ginsel L A, von Recum A F, Jansen J A Orientation of ECM protein deposition, fibroblast cytoskeleton, and attachment complex components on silicone microgrooved surfaces, J Biomed Mater Res. 1998, 40(2), 291.
5. Fujisawa N, Odell R A, Poole-Warren L A, Bertram C D, Woodard J C, Schindhelm K, Acute cellular interaction with textured surfaces in blood contact, J Biomed Mater Res., 2000, 52(3), 517;
6. Wilkerson, W. R. et al., Bioadhesion studies on microtextured siloxane elastomers, Polymer Preprints, 2001, 42(1), 147;
7. Gary, B L, Lieu, D K, Collins, S D, Smith, R L, Barakat, A I, Microchannel-Based Platform for the Study of Endothelial Cell Shape and Function, Biomedical Microdevices, 2002, 4(1), 9;

8. Anderson J S, Price T M, Hanson S R, Harker L A, In vitro endothelialization of small-caliber vascular grafts, Surgery, 1987, 101(5), 577;
9. Lee J H, Lee S J, Khang G, Lee H B, The Effect of Fluid Shear Stress on Endothelial Cell Adhesiveness to Polymer Surfaces with Wettability Gradient, J Colloid Interface Sci., 2000, 230(1), 84;
10. Pu F R, Williams R L, Markkula T K, Hunt J A, Effects of plasma treated PET and PTFE on expression of adhesion molecules by human endothelial cells in vitro Biomaterials., 2002, 23(11), 2411.
11. Krijgsman B, Seifalian A M, Salacinski H J, Tai N R, Punshon G, Fuller B J, Hamilton G, An assessment of covalent graffing of RGD peptides to the surface of a compliant poly(carbonate-urea)urethane vascular conduit versus conventional biological coatings: its role in enhancing cellular retention, Tissue Eng., 2002, 8(4), 673;
12. U.S. Pat. Nos. 6,551,838; 6,537,256, 6,491,666, and 5,797,898;

While the invention has been described in detail and with reference to specific examples thereof, it will be apparent to one skilled in the art that various changes and modifications can be made therein without departing from the spirit and scope thereof.

1. An implantable device comprising:
a surface containing a plurality of first zones and a plurality of second zones depressed relative to the first zones so as to provide valleys below a plane defined by the first zones, wherein a first zone to second zone width ratio is non-random throughout the device; and
a biologically active agent in the valley,
wherein the device is adapted to be implanted within an organism such that when said surface is subjected to a flow causing a fluid-induced shear stress, the second zone has a reduced level of the fluid-induced shear stress relative to the first zone in an amount adequate to selectively retain the biologically active agent within the valley.

2. The device of claim 1, wherein the first zone to second zone width ratio is less than 1.

3. The device of claim 1, wherein the first zones and the second zones are non-randomly distributed across the surface.

4. The device of claim 1, wherein the reduced level of the fluid-induced shear stress is at least 20% less than a fluid-induced shear stress of the first zone.

5. The device of claim 1, wherein the reduced level of the fluid-induced shear stress is about 20% to about 100% less than a fluid-induced shear stress of the first zone.

6. The device of claim 1, wherein a geometrical shape of each of the first zone and the second zone and/or a depth of the valleys are non-random.

7. The device of claim 1, wherein the depth of the valleys is at least 0.1 μm below the plane.

8. The device of claim 1, wherein the valley is at least one of an open valley, a partially closed valley, and a closed valley. (In one embodiment, the valley has one opening, i.e., partially closed valley. In another embodiment, the valley has two openings, i.e., an open valley, wherein the openings are preferably disposed on opposite ends of the valley, i.e., an open-ended channel. In yet another embodiment, the valley is completely surrounded by at least one wall, e.g., a half of a sphere, or two walls, e.g., a closed ended channel, to form a closed valley (long and skinny).

9. The device of claim 1, wherein the valleys are oriented to be at least one of (1) substantially parallel to the flow, (2) substantially perpendicular to the flow, and (3) disposed at a non-parallel and non-perpendicular angle to the flow.

10. The device of claim 1, wherein the biologically active agent is a cell.

11. The device of claim 1, wherein the device is adapted to deliver the biologically active agent to the organism upon implantation.

12. The device of claim 1, wherein the device is adapted to substantially retain the biologically active agent on the device upon implantation.

13. The device of claim 12, wherein the device is adapted to substantially retain the biologically active agent on the device upon implantation without being covalently bound to the device.

14. The device of claim 1, adapted for use as a vascular graft.

15. A method for producing the implantable device of claim 1, said method comprising:
providing a substrate having a surface;
providing the surface with the plurality of first zones and the plurality of second zones depressed relative to the first zones so as to provide valleys below a plane defined by the first zones,
selecting the first zone to second zone width ratio throughout the device such that when the device is implanted within an organism and said surface is subjected to a flow causing a fluid-induced shear stress, the second zone has a reduced level of the fluid-induced shear stress relative to the first zone in an amount adequate to selectively retain a biologically active agent within the valley, and thereby producing the implantable device.

16. The method of claim 15, wherein the first zone to second zone width ratio is less than 1.

17. The method of claim 16, further comprising selecting a geometrical shape of each of the first zone and the second zone and/or a depth of the valleys.

18. A method for delivering a biologically active agent to an organism, said method comprising:
providing the implantable device of claim 1;
regulating retaining of the biologically active agent on the implanted device by adapting the surface to have areas of a reduced level of a fluid-induced shear stress via a pattern of the first zones and the second zones provided on the surface; and
subjecting the device to a flow causing the fluid-induced shear stress by implanting the device within the organism, wherein the second zones have the reduced level of the fluid-induced shear stress relative to the first zones in an amount adequate to selectively release the biologically active agent on the implanted device and thereby delivering the biologically active agent to the organism.

19. The method of claim 18, wherein regulating comprises selecting the first zone to second zone width ratio, a geometrical shape of each of the first zone and the second zone and/or a depth of the valleys.

20. The method of claim 19, wherein the biologically active agent is a cell.

21. In a method of manufacturing of an implanted device having an outer surface adapted to retain endothelial cells, the improvement comprising regulating retaining of the endothelial cells on the surface by providing the device with areas of a reduced fluid-induced shear stress via a pattern of first zones and second zones provided on the surface, wherein the second zones are depressed relative to the first zones to provide valleys below a plane defined by the surface, wherein the pattern is chosen by selecting a first zone to second zone width ratio, a geometrical shape of each of the first zone and the second zone and/or a depth of the valleys such that the second zones have the reduced level of the fluid-induced shear stress relative to the first zones in an amount adequate to retain the endothelial cells on the implanted device.
